(19) United States
(12) Patent Application Publication
(10) Pub. No.: US 2008/0300475 A1
(43) Pub. Date: Dec. 4, 2008

(54) Laryngoscope with Time Indicating Means and Method for Use Thereof

(76) Inventors: Abraham Jaeger, Ramat Hasharon (IL); Yuval Bar, Ramat Hasharon (IL)

Correspondence Address: SCHWEITZER CORNMAN GROSS & BONDELL LLP, 292 MADISON AVENUE - 19th FLOOR, NEW YORK, NY, 10017, US

(21) Appl. No.: 10/583,437

(22) PCT Filed: Dec. 9, 2004
(86) PCT No.: PCT/IL04/01116

§ 371 Date: Jun. 16, 2006

(30) Foreign Application Priority Data
Dec. 16, 2003 (IL) 159383

(51) Int. Cl. A61B 1/267; G04B 47/00; A61B 5/145; A61B 5/01; A61B 5/08

(52) U.S. Cl. 600/364; 600/185; 368/10; 600/549; 600/532

(57) ABSTRACT

The present invention discloses a laryngoscope (1) comprising time indicating means (20). This medical device comprising a handle (2) having a distal portion and a proximal portion; a blade (3) adapted to be inserted into the respiratory tract of the patient; said blade comprising a distal portion and a proximal portion and said distal portion being in communication with said proximal portion of the handle (2); time indicating means (20) adapted to indicate that medical procedure of endotracheal intubation has been terminated or is to be terminated soon. The laryngoscope may also include sensing means (42). The present invention also discloses a method for endotracheal intubation of a patient by means of the aforesaid laryngoscope (1).

### FIELD OF THE INVENTION

The present invention relates to a laryngoscope comprising an integrated or non-integrated time indicating means, and to a time-indicated method for endotracheal intubating by means of said laryngoscope

### BACKGROUND OF THE INVENTION

Laryngoscopes are used to facilitate endotracheal intubation of a patient to provide a positive air passageway for the administration of anesthesia and/or for the mechanical ventilation of the lungs of the patient.

In the human anatomy, the epiglottis normally overlies the glottic opening into the larynx to prevent the passage of food into the trachea during eating; therefore, in endotracheal intubation, it is necessary to displace the epiglottis from the glottic opening to permit the endotracheal air tube to be inserted into the trachea.

Two general types of rigid blade constructions are the straight, or so-called “Miller blade”, and the slightly curved, or so-called “Macintosh blade”. The Bullard Laryngoscope improves over these prior art laryngoscopes by providing an apparatus permitting the simple and rapid visualization of a target area such as the glottis to guide the insertion of an endotracheal tube.

The procedure of endotracheal intubation usually provided by the following steps:
1. aspirating stomach contents prior to procedure if the patient has been fed recently;
2. positioning the patient's head in the manner that his neck is slightly flexed and his head is extended;
3. passing laryngoscope blade gently along the side of the mouth and gently pull tongue and epiglottis forward by lifting the blade. If the vocal cords and epiglottis do not come into view, pulling the laryngoscope back gradually until they are seen to avoid intubation of the oesophagus; and,
4. immobilizing said laryngoscope to the patient,

It is acknowledged in this respect that if the patient remains bradycardic for more than 30 seconds during the procedure and intubation is not near complete, the tube should be immediately removed and ventilation of the patient must provided, such as by a means of bag and mask.

Many laryngoscopes were disclosed in the art and provided for better visualization of the procedure of endotracheal intubation, e.g., the current location of the laryngoscope blade in the aspiration track. Such for example is U.S. Pat. No. 5,665,052 to Bullard, which presents an intubating stylet and laryngoscope. Those advanced devices are comprised of various means for avoiding damage of the organs and decreasing the intubation time. It is According to the American Heart Association (AHA) that the procedure should not take longer than 30 seconds to complete without oxygenation. All those improvements are not successfully eliminating intubation time to lower than 30 seconds so the serious danger for strangulation and/or asphyxiation in infants, toddlers and trauma patient is yet not eliminate.

### SUMMARY OF THE INVENTION

It is thus one object of the present invention to provide a cost effective and highly useful laryngoscope comprising time indicating means. This laryngoscope comprising a handle having a distal portion and a proximal portion; a blade adapted to be inserted into the respiratory truck of the patient; said blade comprising a distal portion and a proximal portion; said distal portion is in communication with the said proximal portion of the handle; and at least one time indicating means adapted to indicate that medical procedure of endotracheal intubation is been terminated or to be terminated soon.

It is in the scope of the present invention wherein the time indicating means is integrated in the laryngoscope; and/or wherein the said time indicating means is attached to the laryngoscope in a non-integrated manner. The said time indicating means is adapted to display the time remains until the procedure shall be terminated and such a display may be selected from a contentious or a discontinuous display. This time indicating means may be also adapted to display the seconds before termination of the procedure and/or to display the stage of the procedure. This time indicating means may be attached to the handle portion of the said laryngoscope. It may be characterized by a bracelet-like rounded structure adapted to grasp the handle in at least one location. It may alternatively be characterized by a plug-like or a cup-like structure adapted to plug in or to be inserted or to be screwed to the handle.

It is another object of the present invention to provide a cost-effective, disposable or non-disposable time indicating means. This means is adapted to indicate that medical procedure of endotracheal intubation is been terminated or to be terminated soon; said means is attached in an integrated or non-integrated manner to a laryngoscope. The aforementioned time indicating means may also be adapted to display contentiously or discontinuously the time remains until the procedure shall be terminated. This time indicating means may be adapted to display the seconds before termination of the procedure and/or to display the stage of the procedure. It is potentially attached to the handle portion of the said laryngoscope.

It is still another object of the present invention to provide a method for endotracheal intubating a patient by means of a laryngoscope as defined in any of the above. This method is comprised the steps of resetting the means so time count is provided for a predetermined measure; and than intubating the patient in any procedure known in the art; wherein said procedure must be terminated before said means is indicating the said predetermined time period is over. Additionally or alternatively, the present invention provides for a method for endotracheal intubating a patient, comprising the steps of resetting the time indicating means as defined in any of the above, so time count is provided for a predetermined measure; and then intubating the patient in any procedure known in the art; wherein said procedure must be terminated before

### BRIEF DESCRIPTION OF THE INVENTION

In order to understand the invention and to see how it may be implemented in practice, one embodiment will now be described, by way of non-limiting example only, with reference to the accompanying drawing, in which

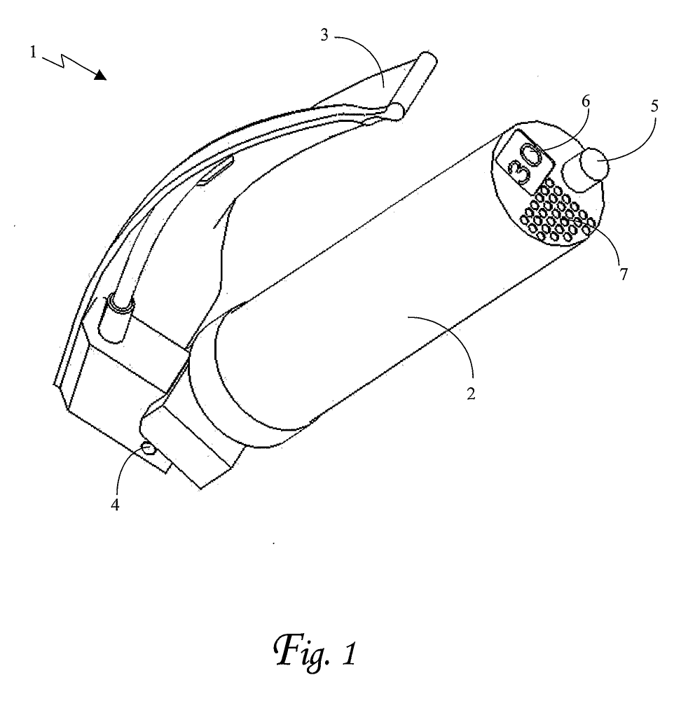

FIG. 1 schematically presents a side view of a laryngoscope comprising an integrated time indicating means;

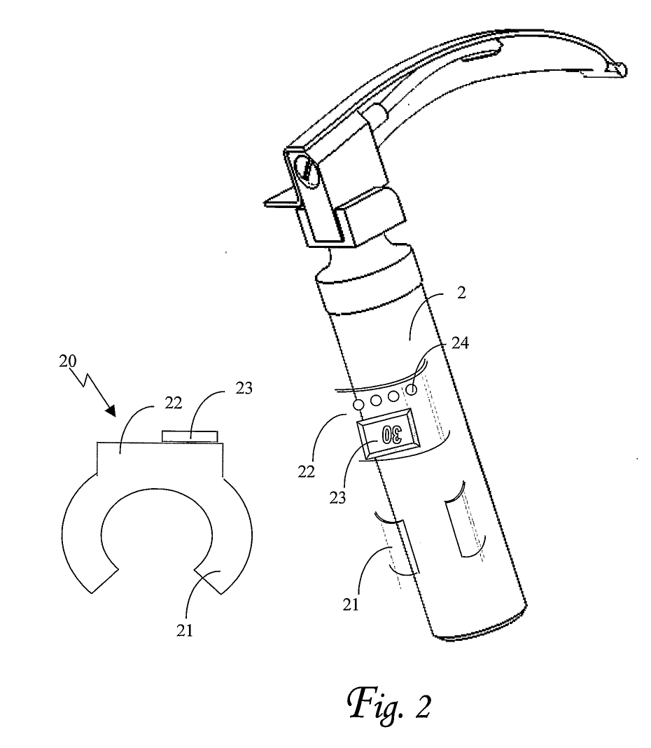

FIG. 2 schematically presents a side view of a laryngoscope comprising a non-integrated time indicating means; and a cross section of the same;

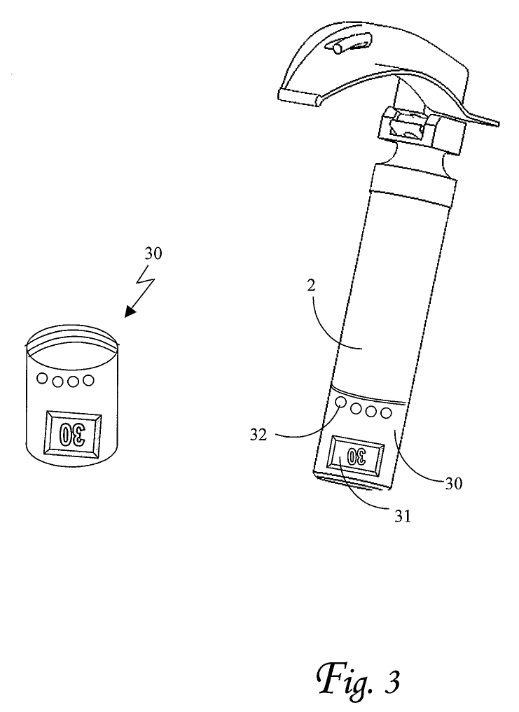

FIG. 3 schematically presents a front view of a laryngoscope comprising an integrated or non-integrated time indicating means and a 3D presentation of the same; and,

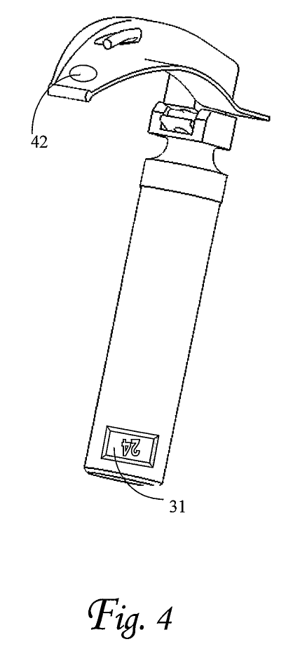

FIG. 4 schematically presents a front view of a laryngoscope comprising sensing means.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a laryngoscope comprising a time indicating means.

The term ‘laryngoscope’ is referring according to the present invention to any medical device adapted to introducing tubes for artificial breathing in air pipes of a patient. More specifically, the term ‘laryngoscope’ is referring to all means adapted for endotracheal intubation, such as in the case of insertion of a tube into the larynx for the passage of a gas such as an anesthetizing gas. This term is equally referring to all types and shapes and sizes, e.g., to laryngoscopes selected for the commercially available Miller, Macintosh or Bullard types etc. This term is also referring to any laryngoscope, which comprise at least in its portion one or more disposable or flexible elements.

The term ‘time indicating means’ is referring according to the present invention to any signaling means adapted to sign, indicate, measure & display, announce or designate the time lest until a predetermined time period is over. Said indication is selected in a non limiting manner from producing visual signs (e.g., light emitting by a means of an array of LEDs, light flashes, displaying seconds to termination etc.); producing audio or vocal signs (such as buzzing, whistling sounds, announcing the time by vocal massager etc); vibrating at least a portion of the handle portion of the laryngoscope or any combination thereof.

The term “sensing means” is referring hereinafter to any sensor adapted to sense a physiological, biochemical or other parameter of the treated patient. The term is particularly referring to a pulse oxymeter for measuring a blood oxygen saturation of a subject; thermometer and CO₂ meter and/or capnograph.

It is according to one embodiment of the present invention wherein the term ‘time indicating means’ is referring to any means adapted to display the time remain until the procedure should be terminated, i.e., according to the AHA no more than 30 seconds. It is according to another embodiment of the present invention wherein the term ‘time indicating means’ is also referring to any programmable means. Hence for example, the means may be adapted to measure and display said 30 seconds, whereat 15 seconds are remaining to termination, a special announcement is provided. Such a display may be selected from any audio, visual or vibrational means. Those visual mans may be selected from lamps, light flashes, white or colored LEDs array, displays adapted to be seen in the dark etc.

It is also according to yet another embodiment of the present invention wherein the term ‘time indicating means’ is also referring to any of the aforementioned means that additionally comprising auxiliaries. Said auxiliaries may be selected in a non limiting manner from acoustical, visual or other recording, transmitting and or displaying of the intubation process. It is still according to yet another embodiment of the present invention wherein said time indicated means are comprised of a mechanism adapted to evaluate and display the number of the intubation attempts provided per time or a total number of those procedures.

It is in the scope of the present invention wherein the laryngoscope as defined above comprising a handle housing and a blade to be inserted to the patient. The handle usually comprised of one or more batteries for energizing a light source associated with the laryngoscope. The blade is adapted in its various types to displace the epiglottis and to provide access to the larynx while passing the tube. The laryngoscope blade also helps keep the tongue out of the way and can be used to apply pressure to the lower jaw for moving the lower jaw during the insertion of the tube. Some of the laryngoscopes as defined above may also be used for the examination of a patient's larynx without intubation.

Reference s made now to FIG. 1, presenting a side view from the back forwards of a laryngoscope (1) in its close configuration. This laryngoscope is comprised of an integrated time indicating means, and is especially useful for indicating the time left for terminating a medical procedure of endotracheal intubation. The laryngoscope (1) comprising a cylindered handle (2) and a beak-like blade (3). The handle (2) is having a distal portion and a proximal portion. Usually said handle is adapted to accommodate batteries for supplying energy for the light source of said scope. The blade (3) is adapted to be inserted into the respiratory truck of the patient. It is comprised a distal portion and a proximal portion; said distal portion is in communication with the said proximal portion of the handle by a means of a hinge (4). According to one embodiment of the present invention, the means for indicating the time are located at the very distal portion of the handle (2) and comprised of a reset button (5), a display (6) and a loudspeaker (7) adapted to produce audio signals. The power source may be selected from either one or more DC batteries, to be in communication with the laryngoscope batteries, to be connected to an AC power source or a combination thereof.

Reference is made now to FIG. 2, presenting a side view of a laryngoscope (1) in its open configuration and a non-integrated time indicating means (20) according to yet another embodiment of the present invention. The aforementioned non-integrated time indicating means (20) is characterized by a bracelet-like structure, adapted to be reversibly attached to the handle (2) by a means of two or more stoppers (21). The inner diameter of said rounded structure is more than a half than the outer perimeter of the handle (2) in it at least one portion. The left hand presentation shows a top cross section of the time indicating means (20). Said means (20) is presented both from its front face (22), and from its rear portion (21). The time indicating means (20) is comprised of a push button comprising a display (23); wherein said button is also adapted to reset the time measurement. This means (20) is optimally also comprises at least one loudspeaker (24). According to this embodiment, the means (20) is adapted to fit with every commercially available laryngoscope having a rounded or polygonal handle (2). This laryngoscope is easily sterilized.

Moreover, this non-integrated time indicating means (20) may be disposable. Hence, it is made of any suitable polymeric compositions, such as thermosetic polymers, high-density polypropylene and/or high-density polystyrene, polyurethane etc. Additionally or alternatively, it may be coated by metal envelope. The power source may be selected from either one or more DC batteries, to be in communication with the laryngoscope batteries, any to be connected to an AC power source or a combination thereof.

Reference is made now to FIG. 3, presenting a front view of a laryngoscope (1) in its open configuration and either a non-integrated or an integrated time indicating means (30) according to yet another embodiment of the present invention. The aforementioned non-integrated time indicating means (30) is characterized by a plug-like shape, which is adapted to plug the distal end of the handle (2), such as in the manner said means (30) are the adapted to be in the shape of the batteries cover of the laryngoscope. Said means (30) comprising a similar push button (31) having a visual indication for the time remain and one or more loud speakers.

Before the sterilization, the means (30) is plugged, twisted or screwed out of the handle, so the batteries are taken off. This time indicating means (30) may be disposable or non-disposable, and may acquire power supply from either the laryngoscope's batteries or form its own battery.

It is acknowledged in this respect that the time indicating means of the first aforementioned embodiment is presenting the displayed time remain until the end of the procedure (6) to the rear portion of the laryngoscope; the time indicating means (20) is displaying the same towards any lateral side and the time indicating means (30) is displaying the same either towards the rear portion and/or towards the lateral face of the medical device.

According to the current procedure, endotracheal intubation shall not be longer than 30 seconds. Hence, the time indicating means (6, 20, 30) are adapted to indicate 30 seconds before signaling that the time has passed.

It is also in the scope of the present invention wherein the laryngoscope comprising sensing means, comprising a handle having a distal portion and a proximal portion; a blade adapted to be inserted into the respiratory truck of the patient; said blade comprising a distal portion and a proximal portion; said distal portion is in communication with the said proximal portion of the handle; and at least one sensor, located at the distal portion of the blade. The sensor is selected in a non-limiting manner from a pulse oxymeter; a thermometer; a CO₂ meter; capnograph or any combination thereof. A feedback is thus provided between the sensor, signaling means etc such that wherein a sensed value is either decreasing or increased a predetermined measure, a signal is provided so that the laryngoscope is immediately removed outside the body.

Reference is made thus to FIG. 4, presenting a front view of a laryngoscope (1) in its open configuration comprising a pulse oxymeter sensor (41) located at the distal portion of the blade, and a screen (31) presenting the oxygen saturation value, such that wherein said value is decreasing a predetermined measure, a signal is provided so that the laryngoscope is immediately removed outside the body. Moreover, a method for endotracheal intubating a patient by means of a sensor-containing laryngoscope as defined above is hereto provided. This method comprising inter alia the steps of intubating the patient in any procedure known in the art; sensing a predetermined parameter, such as oxygen saturation, CO₂ level, organ temperature etc, wherein the procedure is terminated before said sensor is sensing that the aforesaid parameters are exceeding their allowed range. A combined method comprising both time measuring and sensing physiological parameters so that a feedbacked intubation procedure is provided.

1. A self-contained laryngoscope comprising time indicating means for indicating a time associated with an endotracheal intubation procedure , additionally comprising;
a. a handle having a distal portion and a proximal portion; and,
b. a blade adapted to be inserted into a respiratory track of a patient;
said blade comprising a distal portion and a proximal portion; said distal portion Is in communication with the proximal portion of the handle.

2. The laryngoscope according to claim 1, wherein the time indicating means is integrated in the handle of the laryngoscope.

3. The laryngoscope according to claim 1, wherein the time indicating means is attached to the laryngoscope in a non-integrated manner.

4. The laryngoscope according to claim 1, wherein the time indicating means is adapted to display a remaining time for the procedure.

5. The laryngoscope according to claim 4, wherein the remaining time is displayed continuously or discontinuously.

6. The laryngoscope according to claim 1, wherein the time indicating means is adapted to display remaining seconds before a termination of the procedure.

7. The laryngoscope according to claim 1, wherein the time indicating means is adapted to display a stage of the procedure.

8. The laryngoscope according to claim 1, wherein the time indicating means is attached to the handle of the laryngoscope.

9. The laryngoscope according to claim 8, wherein the time indicating means is characterized by a bracelet-like rounded structure adapted to grasp the handle in at least one location.

10. The laryngoscope according to claim 8, wherein the time indicating means is characterized by a plug-like or a cup-like structure adapted to connect to the handle.

11. A time indicating means adapted to indicate a termination time of an endotracheal intubation medical procedure; said means is being adapted to be attached in an integrated or non-integrated manner to a laryngoscope.

12. The time indicating means according to claim 11, adapted to display a remaining time until the procedure is to be terminated.

13. The time indicating means according to claim 11, wherein the time is displayed continuously or discontinuously.

14. The time indicating means according to claim 11, wherein the time indicating means is adapted to display remaining seconds before termination of the procedure.

15. The time indicating means according to claim 11, adapted to display a stage of the procedure.

16. The time indicating means according to claim 11, adapted to be attached to a handle portion of the laryngoscope.

17. The time indicating means according to claim 16, characterized by a bracelet-like rounded structure adapted to grasp the handle in at least one location.

18. The time indicating means according to claim 16, characterized by a plug-like or a cup-like structure adapted to connect to the handle.

19. A laryngoscope comprising non-pressure sensing means, comprising;
a. a handle having a distal portion and a proximal portion;
b. a blade adapted to be inserted into a respiratory truck of a patient; said blade comprising a distal portion and a proximal portion; said distal portion in communication with the proximal portion of the handle; and
c. at least one sensor, located at the distal portion of the blade.

20. The laryngoscope according to claim 19, wherein the sensor is a pulse oxymeter useful for ensuring intubation efficacy by measuring blood oxygen saturation of a subject during said intubation.

21. The laryngoscope according to claim 19, wherein the sensor is a thermometer.

22. The laryngoscope according to claim 19, wherein the sensor is a CO₂ meter or capnograph.

23. The laryngoscope according to claim 19 comprising both time indicating means and sensing means.

24. A method for endotracheal intubating a patient by means of a laryngoscope as defined in claim 1, comprising the steps of resetting the time indicating means to provide a time count for a predetermined time period; and than intubating the patient and terminating the procedure before said means is indicating the predetermined time period is over.

25. (canceled)

26. A method for endotracheal intubating a patient by means of a laryngoscope as defined in claim 19, comprising the steps of intubating the patient; sensing at least one predetermined parameter, and signaling wherein the procedure is terminated before said sensor is sensing that the parameter is exceeding an allowed range.

27. The method according to claim 26, wherein the predetermined parameter is selected from oxygen saturation, CO₂ level, organ temperature or any combination thereof.

28-33. (canceled)

34. The laryngoscope according to claim 1 comprising both time indicating means and sensing means.
